(19) United States
(12) Patent Application Publication
(10) Pub. No.: US 2003/0130580 A1
(43) Pub. Date: Jul. 10, 2003

(54) Sequential stress testing of the heart to indicate metabolically, possible hibernating myocardium

(76) Inventor: Keith E. Kenyon, Van Nuys, CA (US)

Correspondence Address: Keith E. Kenyon, M.D., 14435 Hamlin Street, Ste. C, Van Nuys, CA, 91401-6205, US

(21) Appl. No.: 10/040,871

(22) Filed: Jan. 7, 2002

(51) Int. Cl. A61B 5/02

(52) U.S. Cl. 600/481

(57) ABSTRACT

This disclosure is to enable the metabolic nutrient d-ribose to be administered as a precursor for ATP for diagnostic use both to determine abnormal heart muscle from normal under stress to detect possible hibernating myocardium and to differentiate the degree of abnormality or hibernating when abnormal hearts are discovered by this or other techniques and in addition to enable a practitioner to determine the amount of de novo d-ribose that would make an improvement in the therapy of ischemic hearts.

### RELATED APPLICATIONS

This patent application is related to patent application Ser. No. 09/504,805, “The Use of D-Ribose to Improve Cellular Hypoxia and to Better Absorb Medicaments and Nutriceuticals”, patent application Ser. No. 09/545,121, “The combination of nonliving source physical energy and non-living source chemical energy to maximize the salvage of ATP”, patent application Ser. No. 09/557,470, “The combination of living-source chemical energy in combination with living-source physical energy to potentiate the salvage of ATP”, and “Using de novo d-ribose to spare NAD in the synthesis of ATP”.

### FIELD OF THE INVENTION

This invention is in the field of stress cardiography to both differentiate normal from abnormal myocardium and establish the degree of any hibernation by the using the heart's metabolic tendencies.

### BACKGROUND OF THE INVENTION

If people recover from a myocardial infarction, there is always the question of whether viable but hibernating myocardium still exists in the affected segments. This means that whatever their doctors have done, has enough been done to ensure that there is no remaining viable but inactive myocardium? If it is not discovered in time, there remains the nagging possibility that it will die and become non-viable scar tissue. Unfortunately hibernating but viable myocardium is not always detected in time, as hibernating so possibly still viable, thus preventing timely revascularization, ultimately resulting in scar tissue when not revascularized in time. It is very expensive to detect such hibernation at present and is often not looked for once a patient has “recovered” from the acute episode. Metabolic means to diagnose the heart must use both biochemistry and physics.

Serial testing with diagnostic scanning, the physics, and the metabolic nutrient d-ribose, the biochemical, when used together can make a difference in cost-effective, timely diagnosis and avoiding a number of false conclusions. De novo d-ribose appears to be utilized metabolically only by a heart that is impaired by an ischemic myocardium. It does not appear to be able to be utilized by a normal heart in any way that would improve the normal heart's function. This is proven by the fact that de novo d-ribose cannot enable a normal heart to change the reporting parameters when undergoing testing by the varying means used for common cardiac stress testing, which is to report changes in diagnostic functioning due to the ribose being administered. The tests will remain essentially the same with or without d-ribose. On the other hand, such is not true with ischemically impaired hearts. In other words, if an individual with a normal heart is given a stress test using one of the various means that will be described herein, and the test is reported as it usually is reported for a normal study, it will not change significantly or diagnostically if it is repeated after the administration of de novo d-ribose for a period of time in between the two tests. However, if de novo d-ribose is administered in between the two tests with an impaired heart, there often will be significant differences between the two studies. Therefore, this disclosure will offer a new way to use d-ribose, not necessarily as a substance for nutrition in and of itself as it is used today, but to use it to determine whether or not a heart is normal or abnormal by means either of a stress electrocardiogram or of imaging stress tests. Both types of testing involve scanning of the heart, and such changes, only shown with impaired heats, can vary with the degree of impairment. Scanning the heart from the surface is not considered invasive, and with stress electrocardiography (ECG) the heart can be scanned either intermittently or continually with respect to electrical conduction while the individual being tested is moving on a treadmill, stationary bicycle or two-step platform. On the other hand, stress scans of most handicapped people require inotropic means by chemicals and when magnetic, radiation and even ultrasound are used to obtain images, a stationary thorax is required for best results.

The metabolic nutrient d-ribose is not harvested as an individual molecule in plants, as are glucose and sucrose, but is manufactured by a recombinant DNA process using raw material containing glucose such as corn. The chemical synthesis is provided by the mitochondria of bacteria to remove a carbon atom and render 6-carbon-atom glucose as 5-carbon-atom ribose. De novo d-ribose can be utilized by fatigued skeletal muscles to recover faster by salvaging and synthesizing adenosine triphosphate (ATP) over an 8-hour period instead of the 72 to 96 hours it takes the mitochondria to do so starting with glucose. Normal cardiac muscle does not fatigue in the same fashion, or it would be unable to sustain life. Therefore, although ATP is used by both cardiac and skeletal muscle for metabolic energy, the heart will salvage it rapidly through intrinsic channels even when skeletal muscles can't do so as quickly. If this were not true, cardiac arrest would occur with intensive exercise, and complex life forms couldn't exist.

On the other hand, if the heart is vascularly impaired so that segmental ischemia results, those myocardial segments that become nutritionally and thereby metabolically impaired, lose the intrinsic ability to salvage or synthesize all the ATP they need quickly enough and get to the point that they cannot contract at all and with continued lack of nutrition result sooner or later in becoming scar tissue or irreversible segmental myocardial atrophy. Yet sometimes the ischemia is not complete so the segment is still viable, but cannot salvage enough ATP for normal action. This kind of myocardium is called hibernating, and the heart segments can be saved when identified in time and revascularization employed to restore nutrition and enable their cells to salvage and synthesize ATP in the normal way cardiac muscle cells do. This is usually accomplished by surgery or other invasive means in order to revascularize.

The heart is carditrophic when all segments are fully nourished and cardiatrophic when one or more segments are not fully nourished. De novo d-ribose, being the nutrient precursor for ATP can be utilized by the heart only when a lack of a blood supply interferes with its ability to use the intrinsic normal pathways as the only needed source of salvaging and synthesizing ATP and now will accept extrinsic nutrition for cardiac metabolic energy even if only by tissue perfusion, as far as it will go, if there is inadequate collateral coronary circulation. Viable but hibernating myocardial segments, i.e. reversible cardiatrophic segments, fall into this category, but carditrophic myocardium has no need for outside nutrition and won't use significantly more ATP than the sufficient amount it has intrinsically available. The heart works harder by beating faster and pumping more blood, not by becoming larger and, thereby, stronger as skeletal muscles do. Because they use up ATP differently, normal skeletal muscle can become depleted of ATP by increasing the workload, so will recruit more at any time from any source intrinsic or external. This brings up the problem of dosing d-ribose to go to impaired cardiac muscle during the scans.

Since skeletal muscles will utilize de novo d-ribose as much as they can and use 70% of the body's ATP, in the event that a cardiatrophic heart muscle needs some, there has to be a clear excess of d-ribose administered to make a difference. If cardiatrophic segments get enough of this extra nourishment because a large amount is being administered, even though they have been hibernating because they did not have enough ATP, now they will start contracting, and two things will happen in many cases. The previously hibernating segments will now appear to be contracting on being scanned, and there will be a change in the ability of those segments to conduct electricity. This will show up as improvements in the tracings of at least some of the ST segment deviations and in the contractility of the moving images of the myocardium. If we know what these scans are before ribose is administered and then what they are afterwards, differences can be compared when such dual studies are recorded.

Therefore, to use d-ribose to nourish cardiatrophic segments as a stress-induced diagnostic aid, so as to differentiate them from normal carditrophic segments, requires identification of the problem segments as having problems before the administration of d-ribose. After the administration of d-ribose, if there is a change as a result of better nutrition for more metabolic energy for the impaired heart, there is a diagnostic success in that the heart now may be deemed as a candidate for medical or surgical treatment or further study to determine which or both.

Up until now stress scanning, including ECG stress tests, of the heart using d-ribose have been used to differentiate degrees of impairment in the known cardiatrophic heart to see whether de novo d-ribose would improve heart function. Ribose has not been used to differentiate normal from abnormal—carditrophic from cardiatrophic—for the purpose of simply discovering whether or not a heart is nutritionally impaired, and certainly not in doctors' offices. As a consequence, the investigation of the medical use for d-ribose has been employed with population controls and not with a control on the individual patient by doing a test without ribose, then doing a second one immediately after the administration of ribose for a set period of time, in order to differentiate normal from abnormal. Therefore, it has not been realized before this disclosure that a dual sequential stress test would be needed to detect the differences in nourishment and thereby metabolic activity between carditrophic and cardiatrophic segments or even that such a test was possible or desirable.

Hitherto, since d-ribose is not indicated to nourish normal hearts because it isn't able to, it has only been used to demonstrate that it improved the rate of synthesis and salvage of ATP in diseased myocardium as it did in normal skeletal muscles. In view of this it was suggested that the administration of d-ribose could be used to better determine that there was differential myocardial contractile abnormality by simply giving d-ribose to individuals known to have contractile impairment by other diagnostic means. To use d-ribose metabolically to differentiate cardiatrophy from carditrophy in a doctor's office by stress ECG was not considered. As a consequence, nothing has been done to use d-ribose in a basic test for myocardial impairment by taking advantage of the metabolic way de novo d-ribose functions differently between normal and abnormal. Part of this may be the expense of d-ribose and its limited effectiveness, nutritionally, with the heart as opposed to skeletal muscles. As a consequence, after a decade of using ribose to investigate how it improves contractile heart function from the therapeutic point of view, cardiologists have still not used ribose diagnostically to improve the detection of hibernating but viable cardiatrophic myocardium with greater selectivity, sensitivity and accuracy, and few practicing doctors even know about its therapeutic capability.

However, since it has its own therapeutic physiological affect, if de novo d-ribose were to be used to detect hibernating myocardium and to reduce errors, it would still require that a routine baseline study without de novo d-ribose be done, followed by a study using ribose with and without stress, with stress either physically or by chemicals inotropically induced, in both the baseline and follow-up studies. This is not being done at present to better identify such scar tissue as being still viable so is reversible segmental cardiatrophy. Having a non-surgical therapeutic benefit as well as a surgical one, both determined as a result of the various diagnostic studies presented herein, are the reasons for this disclosure.

First among the equipment to be used to detect normal from abnormal myocardium is the use of the electrocardiogram or ECG which measures the conductivity of the heart rather than the anatomy, and there are variations of the apparatus such as the vector cardiogram. Portable means such as the Holter monitor are included here. In addition, means of non-invasive stress imaging can be used with physical exercise limited, so requiring chemically induced cardiac exercise while testing goes on. This list includes scanning by echocardiographs, thallium scintigraphy, PET (positron emission tomography), CT (computerized tomography), MRI (magnetic resonance imaging) and even electron beam imaging, all under a prescribed load of exercise. These are called stress cardiac studies. Many of these, including PET scans, sometimes error in reporting hibernating but viable myocardium as irreversible scar tissue that revascularization won't help. Such errors will be minimized by using this disclosure. The most cost-effective of imaging scans are echocardiographs, and they will differentiate scar tissue even better with this disclosure,

Unlike other organs, the heart is constantly beating, and fast scanning is needed to image the heart faster than it beats. The fact that the heart beats continually, and the beating is increased by exercise, enables scanning of the heart while exercising to be compared to the heart at rest. The heart is first imaged at rest and then during an established protocol for exercise such as the Bruce protocol. Cardiatrophic scars in the heart such as from a myocardial infarction will often be indicated by a scan during exercise, showing both electrical and wall motion (contractility) abnormalities. Along with the ECG, echocardiography can employ a two-step platform, a treadmill or a stationary bicycle for physical exercise but requires a technician to hold the transducer. It is less costly even though PET and helical CT scanning may be more accurate. Since they are done with the patient more confined, CT, PET and MRI scanning need to use chemical inotropic means such as dobutamine more, but even with echocardiography which can be done in conjunction with a treadmill, the lack of body movement renders improved imaging. Of course, if the patient is unable to exercise at all, regardless of the means used to scan, inotropic drugs are required.

There has been literature reporting the use of d-ribose in energizing cardiac muscle that has been compromised by ischemia resulting in cardiatrophic segments. As a result of this research it has been noted by Pliml, et al. that when ribose in substantial amounts is given to individuals with coronary artery disease, there is identification of nearly twice the number of reversible thallium 201 defects with the use of ribose than with placebo. Pliml was not interested in detecting new defects diagnostically but rather used subjects whose defects were already known in an effort to make angina pain less and reduce ST segment deviation anomalies by administering ribose. Therefore, when he discovered that the start of the deviation of ST segments in the stress ECG's of his subjects was delayed after ribose was used, it was noted as a therapeutic improvement. He did not realize and so did not disclose that if the conduction of the heart were improved because of the administration of d-ribose, previously hibernating myocardium segments that now were viable could have been the reason. He was not interested in the use of ribose nutrition in a doctor's office to diagnose ischemic cardiac segments that were hibernating, so they could be revascularized by surgery as a result of this use. When using dobutamine stress echocardiography (DSE) as an alternative to physical exercise, Gradus Pizlo, et al. noticed that upon infusion with d-ribose compared to placebo, more viable myocardial segments were identified. Stronger wall motion of the heart had been established after the use of ribose than with placebos. He was not trying to find a simple way to detect hibernating myocardium in the first place such as in a doctor's office by screening means, but with already known impaired hearts, identifying as many segments as possible that were hibernating by using de novo d-ribose. The concept of attempting to prove that d-ribose could nourish cardiatrophic myocardium to enable it to function better needed a baseline study just before the ribose is administered or the investigator would be rendered blind to an improvement and the possibility of hibernation not realized.

From the diagnostic point of view carditrophic or normal myocardium is different from cardiatrophic or impaired myocardium in that pathways of metabolism normally closed are now opened to nutritionally impaired myocardium so that these segments will compete now with skeletal muscle for de novo d-ribose, whereas, carditrophic myocardium does not need it, so will not compete because it cannot. Furthermore, clinical research and clinical practicality often follow divergent paths. What works in a research setting involving populations of patients who do not expect results the next day or need immediate surgery, so there is little economic stimulus for quick identification of pathology in these usually chronically ill people, none of whom are emergencies in a clinical setting, does not work in a clinical setting unless the procedures are modified or changed, often considerably, to accommodate the new reality. In a profit-oriented clinical setting it is important to detect hibernating myocardium in a diseased heart as accurately and as rapidly as possible, but it is also important to differentiate normal from abnormal hearts quickly, often after what appears to be an ischemic episode that has just occurred. Therefore, accurate diagnosing needs a quick resolution, and this disclosure seeks to be a way for reliable primary diagnosis before more expensive studies are done. The need for separating normal from abnormal to begin with, followed by differentiating degrees of pathology, is vital because this is an acute vascularly challenged myocardium that may soon lose its viability. If identified accurately soon enough, successful revascularization on a timely basis may be achieved. Accuracy is just as important as speed, and both need to be taken into consideration, but the first need is to tell if a heart is normal or not in the first place. This disclosure offers serial stress ECG separation of normals from abnormals and then with infusion of d-ribose intravenously there can be very rapid differentiating of whether any damaged myocardium is hibernating before invasive procedures.

The fact that these investigative studies date back to 1992, and proposing ribose-enhanced dobutamine stress echocardiographs was reported to the American Heart Association in Atlanta in 1999 with no following clinical implementation, indicates that the medical profession considers DSE to be quite accurate without ribose to differentiate viable hibernating myocardium from scar tissue and have no desire to discover the best way to use ribose as a diagnostic aid or even to use ribose at all, including therapeutically for the heart. They are not interested in using such a simple thing as a stress ECG with ribose nor a DSE with ribose. Since they regard present diagnoses to be sufficiently accurate as is (even if they aren't), they reason that why go to the trouble, taking time and expense, to infuse d-ribose into a patient, if they don't believe it adds anything, even therapeutically, and for all they know, may be detrimental? Actually if not interpreted properly as is being disclosed here or not used at all, ribose or its lack may indeed cause false conclusions as will be explained below. Aside from not using nutrition or ATP at all to determine whether cardiac disease exists, the research has been directed to prove that d-ribose could improve cardiac function in the damaged heart, not diagnose whether the possibility of hibernation exists in a timely way. Thus, when Gradus-Pizlo, et al reported their findings in 1999, their discovery that hibernating myocardium could become more contractile with infused d-ribose fell on deaf ears. Therefore, since cardiologists did not want to use ribose at all, using it as is being proposed in this disclosure was not realized. The equipment may be the same, but the use of d-ribose to discover nutritionally impaired myocardium to find possible hibernation with greater selectivity, sensitivity and accuracy in the differentiation of true scar tissue from hibernating myocardium requires this disclosure.

With repect to false conclusions, using de novo d-ribose without a baseline study may actually be harmful by temporarily improving the energy of the myocardium. A heart scan before de novo d-ribose is administered is needed to compare. Without a routine baseline, if the patient were put on d-ribose for a period of time prior to the scan, and viable-appearing instead of hibernating myocardium were now identified, such scans may be diagnosed falsely as not requiring revascularization, because the apparent viability, showing up as increased wall action is only temporary and just during the test because of the temporary increase in energy—a false normal. Thus, it would not be advisable to use ribose without a baseline. Not using ribose at all as is now the case, may render cardiologists to believe incorrectly that some hibernating myocardium is not viable, when using ribose would show that it possibly could be viable. This sick heart is better than they think—a false conclusion. Then using ribose without a pre-ribose baseline study could result in the heart doing better both in the resting and exercise states and give a false impression that the heart was in better shape than it is, because when the ribose was discontinued the heart would again be less energized and more hypoxic with the cardiologists mistakenly and unknowingly thinking otherwise. This heart is worse than they think—another false conclusion. This disclosure is designed to avert this calamity by using a study both with and without d-ribose to make sure that every patient has the best chance at a conclusive diagnosis so that revasculariztion will be undertaken in every case that it should, and the therapeutic use of d-ribose followed when indicated. A dual sequential protocol is necessary to make the best possible differential diagnosis of scar tissue as soon as possible. Obviously dual sequential stress cardiography, including ECG, echocardiography, CT, PET and MRI scanning, needs to employ d-ribose in the second sequence of the dual scan after enabling a sufficient tissue level of ribose to enable more ATP to be in the myocardium when the metabolism of the heart permits. Such dual sequential scans are best completed within a 24-hour period to protect the patient optimally by time constraints. What is needed to make de novo d-ribose accepted by the medical profession is for the profession to realize that de novo d-ribose does not nourish normal hearts but does nourish ischemic ones. If d-ribose can enable the conduction of the heart to improve, it obviously is nourishing the heart better, so it may signal that without d-ribose being administered some of the myocardial segments may be hibernating. Therefore, by performing the simplest such test, the stress metabolic ECG, to discover if the conduction improves with d-ribose improving metabolism, there may be hibernating myocardium if it does. The same is true with metabolic imaging studies. When one has had a coronary, a most important thing to discover is possible viable myocardium that is now hibernating. The only way to discover viability early is by cardiac metabolism.

This invention is designed to overcome the deficiencies of previous applications and inventions by employing a simple way to determine the presence of abnormal myocardium by differentioning normal from abnormal and whether or not there may be hibernating myocardium by administering metabolic cardiac nourishment, de novo d-ribose, to impaired hearts to see if their function under stress is improved and for abnormal myocardium to determine the degree of malfunction and whether or not there is a therapeutic approach available.

### SUMMARY OF THE INVENTION

Much research has been done with respect to using the nutrient, d-ribose, in order to provide 5-phosphoribosyl-lpyrophosphate or more simply expressed, phosphoribosylpyrophosphate (PRPP), more quickly. By providing de novo d-ribose for at least one half hour, a pronounced stimulatory effect on PRPP synthesis occurs, eliminating much of the time needed for the Dickens shunt, thus, in turn speeding up the pentose phosphate pathway that leads to the synthesis of PRPP and ultimately ATP. De novo d-ribose has been used by researchers in various kinds of stress myocardial studies including graphics and imaging with good scientific results, but they do not take into consideration that the normal heart cannot use de novo d-ribose and does not benefit from any long-term administration of ribose. On the other hand, diseased hearts need a great deal of d-ribose, which has cost problems. Therefore, the substance has been used mostly to facilitate the salvage and synthesis of ATP in fitness or athletic settings, and it has only been used in research as part of evaluating diseased hearts, not differentiating normal ones, because it has not been realized from the diagnostic point of view that normal hearts will remain exactly the same no matter how much stress or de novo d-ribose they are given. Since it takes considerable ribose to be sure that while a normal heart won't accept it, there is enough for an impaired heart to be able to. This adds an expense to a test, but if it can make it possible to select hearts that may have hibernating myocardium, it is well work dual testing. Therefore, if cardiac ischemia is suspected or if present by evidence of conduction abnormalities, the dual study needs to be done. If ST any segment deviation is improved by d-ribose, hibernating myocardium is a possibility, and a complete workup can be done with scanners, once again using a baseline non-ribose study followed by a workup with d-ribose as is being disclosed here.

If de novo d-ribose improved myocardial function temporarily, it would mean an ischemic or cardiatrophic heart could be distinguished from normal or carditrophic hearts. Even as a test to determine the degree of abnormality, the few doctors familiar with ribose did not feel it was worth the effort to use de novo d-ribose to discover hibernating myocardial segments that in conventional solo testing would be visualized as permanent scar tissue. With respect to stress ECG, dual testing or even serial single stress testing as a screen to differentiate normals from abnormals was also not realized to be advantageous. Even that myocardial segments could now change and be scanned as more contractile by d-ribose, was not considered important. The fact that only cardiatrophic segments can utilize de novo ribose because the normal salvaging mechanisms for the heart have become impaired due to the cardiatrophy was not appreciated. Making it more likely for viable myocardium to be accurately differentiated from nonviable myocardial scar segments, so much needed revascularization could be done, was also not appreciated. Nevertheless, the cost of differentiating cardiatrophic from carditrophic myocardium can be low if a Holter monitor with or without modified software is used to determine ST segment deviation under stress. On the other hand, non-contractile segments must be identified in advance as well as ST segment deviation including intervals, before the de novo d-ribose is administered, so the ribose will not mask the abnormal findings by energizing cardiatrophic segments to make them look more normal and fail to identify abnormality. De novo d-ribose is temporarily rapidly therapeutic in cardiatrophic heart muscle in the protocol doses of from 12 to 60 grams in a day. Baselines must be estalished without d-ribose, because cardiatrophic segments must be identified. If revascularization is delayed by failure to do a baseline scan, it will do the patient little good if de novo d-ribose enables damaged cardiatrophic tissue to contract temporarily and by that thought to be carditrophic when it was in fact now unidentified hibernating cardiatrophic myocardium causing doctors to make mistakes, only because a complete baseline study was not done.

Therefore, it will be dangerous for the patient, if d-ribose should be used without such a baseline scan done just before ribose ingestion or infusion is started, which administration should immediately follow the baseline scan, so the overall time of testing is minimized. If surgical alternatives become discarded when they are actually necessary, because a dual sequential test was not done quickly like over a 1 to 24-hour total period, the cardiologist will be held responsible for failure to identify hibernating but viable myocardium in time. The fact that a cardiatrophic heart will utilize d-ribose but a carditrophic heart won't, will enable a number of stress cardiac tests to give valuable results at low cost. On the other hand, in addition to being able to differentiate normal from impaired myocardium at an early point in the disease, this invention may enable many early silent infarctions to be detected as cardiatrophic before any hibernating myocardium becomes permanent scar tissue by using serial stress ECG's, fast imaging scanners and even inotropic drugs like dobutamine with a dual complete study, with and without d-ribose. Hibernating myocardium may be lost in the overall scan of the baseline study and, as a consequence, be ignored, but when it would appear differently on the ribose-protocol part of the study, it would then have attention called to it when otherwise it might not have. Thus, early surgical revascularization to bring about carditrophy for that segment, may be considered when it otherwise would not until later, with the infarct area possibly spreading as more tissue becomes cardiatrophic in the meantime. Even with modem noninvasive scans, permanent scar tissue must be differentiated from viable although hibernating myocardium, and using their metabolic differences is vital for maximum success and must be followed if every means to do so is attempted.

Furthermore, if this more sensitive, valuable information can be uncovered within 24 hours, more timely surgical procedures could be done more often to protect the viability of the hibernating part of the myocardium, and do them better than if the present less sensitive diagnostic routines were followed. When required in order to determine if there is need for rapid surgical intervention, the time interval between the dual successive complete scans could be reduced to as little as 1 to 4 hours using intravenous infusion of d-ribose during the interim between the two tests or following an 8-hour intervening period during which d-ribose was administered by mouth, more timely discovery of hibernating cardiatrophic but sufficiently viable segments to become carditrophic could result, enabling surgical intervention to be enacted sooner. For most cases, if the d-ribose were administered over approximately a 24-hour period to enable more PRPP to be synthesized into ATP and more ATP salvaged, the overall time needed would not delay too much a possibly more accurate diagnosis in most cases. A complete dual sequential stress cardiac scanning has the non-exercise part of the study without de novo d-ribose done first with the exercise part of the scan without ribose following. If the results are negative, there may be no point doing the ribose protocol, but if there is an abnormality, then the 24-hour protocol should begin. The de novo d-ribose is administered to the patient from the moment the first stress study was completed and continued to be administered to the patient until the same time the next day, when a repeat of the same scanning procedure done the previous day is completed. Now a comparison of either electrical conduction or myocardial contractibility of the ribose-protocol part of the test with that of the baseline part the previous day, both sequences being done as close to the same time period with the same environmental status as possible, will be useful in order to distinguish segmental myocardium with viability from that of nonviable scar tissue by these metabolically differentiated scanning procedures.

When the device used is the ECG and screening-serial-stress ECG's without ribose are done at places like health and fitness clubs, and evidence of a recent ischemic episode such as changes in the ST segments appears on the record of a client, ribose can then be administered to that individual and if the ST segments are improved toward normal, it becomes indirect evidence that there is possible hibernating myocardium. Since this myocardium can lose its viability, the possibility of hibernating myocardium can be detected by a common, cost-effective means of diagnosing the ischemic heart with its conduction deficits under stress, now leading the way more quickly to other types of scanning such as imaging, can save lives and reduce morbidity. On the other hand, if ribose enhanced the recovery for diagnostic reasons, it can also enhance the recovery therapeutically and keep this cardiatrophic segment more carditrophic until surgery restores the normal vascularity. Therefore, the continued use of d-ribose during the revascularization procedure may accompany the surgical intervention, since it will render the heart more resistant to temporary ischemia during the procedure. Then, of course, the d-ribose could be continued post operatively in those cases where it was deemed useful in the diagnostic procedure, because the previously impaired heart still needs as much energy as it can get, and with optimum energy of the myocardium, the chance of long-term survival will be improved.

The features of the present invention which are believed to be novel are set forth with particularity in the appended claims. The present invention, both as to its organization and the manner of operation, together with the further objects and advantages thereof, may be best understood by reference to the following exemplary and non-limiting detailed description of the invention.

### DETAILED DESCRIPTION OF THE INVENTION

The following description of dual sequential scanning of the heart is designed to differentiate normal from abnormal myocardium and hibernating but viable myocardium from nonviable scar tissue with greater sensitivity, specificity and accuracy in the suspected ischemic heart by proceeding with baseline rest scans of myocardial electrical conduction, imaging or both, using high-tech scanners, including but not limited to electrocardiography, echocardiography, PET. CT, or MRI electron beam imaging scans without d-ribose being administered for the rest baseline. This rest episode is followed by an exercise baseline study, in order to discover whether there is any abnormality and if so have a basis on which to compare. If the exercise sequence is normal the test is over. If not de novo d-ribose is then administered over a given time period in order to biochemically shorten the time for ATP to be synthesized or salvaged so as to make more ATP through its metabolic pathways available to the hibernating cardiatrophic myocardial segments. Then the complete study is repeated in order to contrast the follow-up result with the baseline study without the nourishment provided. De novo d-ribose is administered to the patient following the baseline study for at least a 1-hour period to as much as a 24-hour period or even longer period, using either infusion or ingestion. The d-ribose having been started immediately following the completion of the first or baseline procedure, the follow-up procedure is done under as close to the same environmental conditions as possible and usually approximately from 1 to 24 hours later, having from 12 to 60 grams of d-ribose administered in divided doses to the patient during the interim period with as much additional d-ribose administered during the exercise part of the second or ribose-protocol part of the test so that as high a level of de novo blood-ribose and by that tissue-ribose as is reasonably possible can be available for the heart at the follow-up testing.

It requires the first step of providing cardiac scanning equipment deemed necessary for the specific objectives of the test to include electrocardiographic as well as imaging means designed to make a record of the beating heart from sequential images by ultrasonic, radiation, magnetic or sequential graphs by conduction means. Conduction means can use a potentiometer with a movable stylus or recorders employing solid state electronics to measure the electrical conduction such as but not limited to, conventional ECG machines such as the HP Page Writer series or the Zymed family of stress ECG recorders including using its Holter software for Windows. The selected equipment from step-1 is used in step-2 for the baseline test during which the patient first lies quietly for the resting part of the study, then exercises by any means deemed appropriate by the individual conducting the test or the limitations of the scanning or conduction means, with the diagnostic equipment either attached to the patient or at the ready for immediate use. With table scanning, inotropic drugs are better used for stress, but when physical exercise is used such as with echocardiography and/or ECG, a treadmill employing the Bruce protocol can put the heart into an exercise mode, but any means, including the two-step platform, to enable the patient to exercise in a convenient manner while enabling successive graphs or images of the myocardium to be made while the patient is exercising, are acceptable. Electrode means for recording by electrocardiographic and sphygmotonographic means should be attached for general monitoring and also because cardiatrophy can be partially identified by indicating a change in the ST segment deviation analysis and other deviations after d-ribose administration. Carditrophic myocardium doesn't need and cannot use the extrinsically administered d-ribose nutrition to improve cardiac metabolic energy so cannot show a stress induced abnormal deviation in ST segment analysis whether or not d-ribose has been administered. Changes in the scanning results after ribose administration thus being limited to cardiatrophic hearts occur both with imaging and graphic scans.

Upon completion of step-2 a full record for the equipment used becomes available on how the patient's cardiac muscle responds to stress without de novo d-ribose being present and if abnormal, the possibility of hibernating segments being present can be shown. Step-3 is either to provide infusions of d-ribose for 1 to 4 hours during which they may be continued during the second sequential or follow-up test, or if infusing ribose is not done, the patient is provided 12 to 60 grams of d-ribose each 24 hours to be self-administered in divided doses. More than 60 grams a day may be taken, but it may cause excess gastro-intestinal symptoms, and 60 grams is enough for a 24-hour period. On the other hand, a total dose of 12 grams a day divided into separate self-administered doses is the reasonable minimum needed to provide successful ribose data. For a 24-hour protocol, when testing is done in the morning, the second dose is taken 4 hours later, as is the third in 4 more hours and the last before going to bed. Another dose is given when the patient returns to the same clinic or hospital at the same time approximately 24 hours after step-2 was initiated, for step-4 to begin. For later starting times the times for taking the ribose may be adjusted. For maximum accuracy the same ambient temperature is maintained, and the same equipment or its equivalent is provided. Step-4 is a repeat of the testing done in step-2 and is accomplished the same way and during the same time period. A fifth dose of ribose may be given at the time step-4 is conducted, once in its entirety, or half may be given at the start of step-4 and the other half, midway through the exercise or stress part of the scan to keep cellular ribose maximum. Then the data acquired from step-2 is compared with the data from step-4 to see if any myocardium previously identified as hibernating is now contracting again and whether there is greater wall action and strength. If taking de novo d-ribose enables hibernating myocardial segments to contract, the segments are at least more viable temporarily, but revasculariztion may still need to be done soon. Ribose may be maintained now for therapeutic purposes, to see the heart through the interval of time needed for surgery to be implemented and beyond if desired. In the case there are no changes after 24 hours, but there is still a strong suspicion that the heart is abnormal or that hibernating myocardium can still be discovered, the ribose may be continued for as long as a week and a follow-up study done. If the test has a normal baseline and a normal follow-up 24 hours later after administering d-ribose, it is usually sufficient to confirm a carditrophic heart.

Conventional ECG equipment, such as Philips Medical System's HP Page Writer series can be used as well as Holter monitor means, including Philips Med. System's Agilent Technology Division's Zymed Holter recorders, with or without their technical suite and with or without software for Windows (Zymed 1810 series including 1810 with Technical Suite and their successors). The latter could be made to be quite effective in health and fitness clubs as well as in doctors' offices as described above, as a screening test, but the procedure may be modified if using the Holter means when such a recorder is not to be worn as designed, continuously. An algorithm will be described both for continuous and sequential use of the Zymed Holtor monitor, but modified software for the Holter monitor needs to be used in health and fitness clubs for screening purposes.

When a Holter monitor is used conventionally there is no sequential separation of the scanning procedure itself, as it is continuous. However, there can be a separation by the metabolic use of d-ribose. This offers the opportunity to determine on a continuous scanning basis whether or not the administration of d-ribose after the scan has started makes a difference in a given defect by improved cardiac metabolism so that such tracings as ST segment deviations improve as the tracing continues. A long baseline tracing followed by an even longer use of the metabolic nutrient ribose enables the tissue level of ribose to rise gradually as more de novo d-ribose is ingested, and changes in the graphic record with such increases in tissue ribose are recorded. The conventional Bruce protocol can be done without ribose and then with ribose, but if this is impractical, repeated two-steps can be used or specific off-site exercise prescribed before ribose is ingested and then repeated after ingestion as often as the doctor prescribes. The recorder's leads are applied to the patient followed by a few minutes of baseline done at rest. Then the Bruce protocol or other stress means are followed without ribose until the baseline exercise protocol is completed and for the amount of time the doctor wishes. The recording starts and continues without ribose, with 6 to 12 hours of daytime activity being reasonable. Following this, d-ribose is administered over a period of up to 24 hours or if desired until the intrinsic recorder storage disc is full or an attached cassette disc or tape is full. Before the storage or the prescribed d-ribose runs out, the final exercise regimen is completed.

To do the test, the unit is applied to the patient, preferably in the morning and after a short rest segment if desired, the patient undergoes normal or prescribed intense exercise for a given period without ribose that does not need to be longer than 12 hours if from 36 to 48 hours are the limits of the recorder. Following this for the next 24 hours up to 60 grams of d-ribose are self administered in divided doses, 15 grams of ribose being taken at the 6 to 12 hour mark and 15 grams just before the final exercise segment is started. If the tracings are changed and improved upon after d-ribose has been administered, it is evidence that hibernating myocardium may be present as a result of the improved cardiac metabolism and revascularization is possible.

Holter monitor means can be modified to replace the conventional ECG in places like health or fitness clubs in order to diagnose serially the normal heart as being normal in a cost effective screening test. This could not ordinarily be done in a doctor's office because space and cost restraints would prohibit it. The rational for this kind of screen is that an individual who produces a normal resting ECG has a test of little value if subsequent exercise, not done, would change it to abnormal. A stress ECG turns up impending ischemia because of the greater demand for cardiac metabolic energy during exercise. Since the heart commandeers all it needs intrinsically when healthy but not when impaired by coronary stenosis, impending stenosis may be indicated first by serial stress ECG's. Such serial screening stress ECG's will never be practical in the average doctor's office, so will not be much available to the public, but this is not the case with respect to the average fitness or health club, but to use such a facility, the algorithm must be modified.

It is disclosed that we rewrite Windows software, so that the continuous and thereby uninterruptedly running algorithm as presently used by Zymed and all other Holter monitors is fundamentally changed to program a new, much more inexpensive per screen, sequential algorithm, each sequence being for a different individual rather than the present continuous one for the same patient, achieving a new mass stress screening use for a Holter monitor. Computer means capable of accessing the Internet are needed for the fastest response. This new software only needs to be designed to report normals or abnormals as one word. Although the software can be written as complicated as desired, it only needs to identify the presence of, but not differentiate, a single abnormality such as QT interval abnormality, ST-T wave deviations, ectopics or arrhythmias and not report abnormals other than as an abnormal stress ECG. The computer can always provide the entire tracing undiagnosed for a doctor to read or to another computer programmed to make a complete diagnosis.

Since the Zymed Holter monitor can be operated continuously for 48 hours of recording, over 200 separate stress-screening sequences of separate individuals can be done with one internal memory chip or one detachable cassette recording means for retrieval and storage. Each segment of a new individual can be separated from the preceding one by software switching means and each segment identified as to which individual is being scanned by keyboard or voice activated means or both. Since abnormal ECG's are quickly and accurately identified by computer software means, and only the word normal or abnormal need be cited with an optional printout, screening costs can be so low that there is no obstacle to screening the entire vulnerable population serially even multiple times a year. Therefore, this test becomes somewhat analogous to the miniature chest X ray, first used mostly to screen tuberculosis, that was only reported as normal or abnormal, relying on private doctors to do a full-scale X ray for diagnosis. To do such screening-stress ECG's one after another in doctors' offices is not practical just like the chest X-ray screenings. However, a Holter monitor is a solid state battery operated recorder that can be worn while a person is on a treadmill or stationary bicycle at a fitness or health club, and it has every bit as much legal right to be used there without a prescription as do pulse or blood pressure recorders or the treadmill itself, since a stress ECG is not invasive by either radiation, as a chest X-ray is, or by ultrasound and does not need to be connected to an electrical outlet as the others are.

Nevertheless, medical legal restraints must be considered. So storage of the signal may be required for a period of time. Costs are least when the computer just reads normals and reports everything else as abnormal without text printouts on paper or individual formatted discs. If a printout or disc is desired, normals can be printed at the treadmill location. Abnormals should only be printed out at a private location with federal HIPAA privacy security, and since this is only a screening test, with abnormals requiring immediate additional studies under more controlled conditions, including using d-ribose, the software does not need to be encumbered with tedious writing about which kind of abnormality is being encountered, only that it is not normal, so the software and any required storage can be least expensive. Differential software writing is already available with more sophisticated equipment that would need to be used anyway to determine the kind of pathology. If such screening stress tests were conducted as often as once a month at a fitness or health club gymnasium and any abnormals reported expeditiously, revascularization would much more often be timely and preventive and peace of mind for the normals greater. Even less frequent testing would be advantageous over present procedure. Therefore, we would combine steps-1 and 2 as just described, using Holter monitor means such as the Zymed 1810 series with our modified software using the appropriate Windows or its equivalent, and serially repeat the screening at a reasonable frequency doing steps-1 and 2 serially on a single individual over time and only do all of the steps-1 through 4 above using the Holter but including as indicated more sophisticated means when Holter monitor abnormals are detected by one of these screenings.

The treadmill means or any other exercise regimen provided in step-2 may be substituted by chemical means to stimulate the heart inotropically while the body as a whole remains at rest, and such means are usually needed for table or platform scanning such as with PET, CT or MRI scanning. The commonest type of inotropic chemical is a derivative of the neurotransmitter, dopamine, and called dobutamine. In the event chemical means to induce contractility of the heart are used, such must be titrated in step-2 by intravenous infusion, and again so used in step-4. The ribose intake remains the same with the same overall time frames for this inotropic dual sequential study. For safety purposes, the blood pressure and heart rate need to be monitored when dobutamine is given, even as the cardiac impaired also should use them with strenuous physical exercise. The Holter monitor means as discussed above can be used with dobutamine to induce stress also, but more sophisticated electrocardiographic means would likely be utilized here since inotropic means would not be done ordinarily in fitness and health clubs but rather at doctors' offices and hospitals on tables. Using dobutamine for a stress metabolic ECG would have more of a use than for an ordinary stress ECG since more diagnostic information would be obtained.

In the event that exercise is not tolerated either by chemical means or physical, the dual test is still done the same way over the same time periods using the same amount of de novo d-ribose, only the exercise part is omitted. Also when only a single episode of exercise is desired do to limited tolerance by the patient, the exercise part of step-2 can be eliminated but not the exercise part in step-4 for maximum diagnostic capability. The inotropic means will be less dangerous to the more energized heart using d-ribose. This will give reliable information without requiring both exercise episodes when minimal exercise is indicated. In either of these alternatives, viewing the heart both before ribose and afterwards will uncover more viable myocardial scar tissue that would not be uncovered if only the non-ribose testing were done, because using de novo d-ribose in testing after its non-use in the baseline part increases the sensitivity, selective-capability and accuracy of the testing. On the other hand, in the event that the individual was given de novo d-ribose and a diagnostic study done, the ribose could be withdrawn and the baseline test done after the fact. Since ribose is used up rapidly, it would not take long for the ribose to be completely metabolized, but at least a day should be allowed for it to stop its effect.

Finally, because of the nature of d-ribose, it is one of a few substances that can be used diagnostically in imaging procedures as well as has a therapeutic use also. Ironically dobutamine is also one of these substances, because it has a medical use of increasing the contractility of a weak heart by neurotransmitter means as well as to enable avoiding physical exercise in imaging. Nevertheless, dobutamine has very limited therapeutic use and only as a short-term therapeutic agent, because it quickly reaches a dangerous level of toxicity. On the other hand, d-ribose increases the contractility of the heart by the metabolic means of providing more energy, and being a basic molecule in both the structure and function of the body, has a very low level of toxicity. Therefore, if it is discovered as a result of this dual scanning using the de novo d-ribose-induced metabolic pathway in the second part, that the heart is more energized with less hibernating myocardium after ribose is taken than before, it stands to reason that it would be useful to have the patient continue to take ribose. Unfortunately ribose is quite expensive to use on a continuing basis in the amounts needed for a heart that needs more ATP but is too ischemic to provide it. The skeletal muscles and the brain all want extra ATP so compete for de novo d-ribose. Therefore, if the ischemic heart is to get its needed share, ribose must be given in large amounts of as much as 60 grams a day. Since ribose costs about 10 cents a gram in large wholesale quantities, this much ribose could cost a patient as much as $20 to $30 a day in individual packages. It might be worth that much money if it could be demonstrated conclusively that de novo d-ribose actually improves the contractility and viability of an individual heart with chronic ischemia. Even so, less of it may work well enough on some people. Since contractile capability can be visualized by scanning, knowing for sure the optimum dosage would make it more cost-effective on the long tern. Once the need for d-ribose is established by the initial test, even after surgical revascularization is done, periodic serial scans, each following a longer than 24-hour period, to establish any new amount of ribose to optimize strength of contractility would follow. As a unique consequence, the dual nature of the test makes it both diagnostic and therapeutic, since it diagnoses hibernating myocardium and then effects a therapeutic improvement by the very substance, d-ribose, which facilitates the diagnosis. Both stress echocardiography of the heart and stress ECG can establish the optimum or minimum dosage of d-ribose continually needed because without enough ribose and no surgical revascularization, the heart would revert to its pre-ribose condition.

While particular embodiments of the present invention have been shown and described, it will be obvious to those skilled in the art that changes and modifications may be made without departing from my invention in its broader aspects of a method to utilize de novo d-ribose to make diagnosing isehemic segments of the heart more sensitive, selective, accurate and earlier with respect to viability of myocardial segments so as to better diagnose the need for surgical intervention and to prove in each case what benefit the therapeutic use of d-ribose is and its optimum or minimum dosage for that individual.

I claim:

1. A method to perform dual, sequential diagnostic testing of the heart on the same patient, with each half of the dual testing having two parts, the first part being a baseline study and the second part being the use of stress means designed to exercise the heart during the second part of the initial half of the dual test, and immediately after its completion, de novo d-ribose is administered for one hour or longer, whereupon, the same two-part test is repeated as the second half of the dual test.

2. The method of claim 1 in which from 12 to 60 grams or more of de novo d-ribose is administered by mouth following completion of the initial half.

3. The method of claim 1 in which stress can be elicited by physical exercise to induce the heart to contract more rapidly.

4. The method of claim 1 in which stress by chemical inotropic means can be used to induce the heart to contract more rapidly.

5. The method according to claim 4 in which dobutamine is the chemical agent.

6. The method of claim 1 in which more than 60 grams of d-ribose are administered during and after the test.

7. The method of claim 1 in which the various stress scanning tests of the heart include but are not limited to electrocardiographs, echocardiographs, thallium scintigraphy, PET (positron emission tomography) scanners, CT (computerized tomography) scanners and MRI (magnetic resonance imaging) scanners and electron beam imaging scanners.

8. The method of claim 7 in which electrocardiograph and sphygmotonograph electrodes are attached to the patient and used for monitoring purposes.

9. The method of claim 1 in which intravenous infusion of d-ribose is used for at least one half hour.

10. The method of claim 1 in which the heart function having been improved diagnostically by de novo d-ribose, said d-ribose is continued therapeutically afterwards.

11. The method in which the minimum practical levels of de novo d-ribose dosage is determined by serial imaging studies, each following the other by more than 24 hours, showing the degree of myocardial contractility for a given dosage of d-ribose, for which any non-invasive, immediately sequential imaging procedure for the heart can be used.

12. The method of claim 7 in which the determination of the heart rate and blood pressure is done manually.

13. The method of claim 7 in which the Philips Medical Systems' electrocardiographs are used for the testing.

14. The method of claim 7 in which Holter monitor means are used as conventionally used on only one person for 24 to 48 hours.

15. The method of claim 13 in which the Holter monitor is one of the Zymed 1810 family of recorders using Windows.

16. The method of claim 13 in which said scanning is done at fitness and health clubs.

17. The method of claim 1 in which when said baseline scanning is reported as normal, the baseline is repeated serially until an abnormality occurs and then the d-ribose protocol followed.

18. The method of claim 14 in which when the Holter monitor is not used as conventionally used, the software is written for intentional sequential interrupting of the scanning so that multiple individuals can be scanned on one unit for recording, retrieval and storage over an elapsed time period that could last up to 48 hours of total although continually interrupted use.

19. The method according to claim 1 in which the ribose part of the test is done first and the baseline afterwards.
